(19) United States
(12) Patent Application Publication
(10) Pub. No.: US 2006/0272107 A1
(43) Pub. Date: Dec. 7, 2006

(54) Hair relaxing composition comprising at least one non-hydroxide polyguanidine

(76) Inventors: Gerard Malle, Villiers S/Morin (FR); Christian Blaise, Saint Mande (FR); Xavier Radisson, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/435,144

(22) Filed: May 17, 2006

Related U.S. Application Data
(60) Provisional application No. 60/685,868, filed on Jun. 1, 2005.

(30) Foreign Application Priority Data
May 17, 2005 (FR) 05 51274

(51) Int. Cl. A61K 8/00

(52) U.S. Cl. 8/405

(57) ABSTRACT

The present disclosure relates to a ready-to-use cosmetic composition for relaxing keratin fibers in a cosmetically acceptable medium comprising, as an active relaxing agent, a polyguanidine not belonging to the hydroxide family. The present disclosure is also directed to a multi-compartment kit comprising at least two compartments to be placed in contact to form the ready-to-use composition. Another aspect of the present disclosure relates to a process for using the presently disclosed composition.

This application claims benefit of U.S. Provisional Application No. 60/685,868, filed Jun. 1, 2005, the contents of which are incorporated herein by reference. This application also claims benefit of priority under 35 U.S.C. § 119 to French Patent Application No. FR 05 51274, filed Apr. 17, 2005, the contents of which are also incorporated herein by reference.

The present disclosure relates to a ready-to-use cosmetic composition for relaxing keratin fibers, comprising, as an active relaxing agent, a polyguanidine not belonging to the hydroxide family. The present disclosure is also directed to a kit comprising compartments to be placed in contact to form the ready-to-use composition, and also to a process using this composition.

As used herein, the term “polyguanidine not belonging to the hydroxide family” embraces polyguanidines and their organic or inorganic salts which do not contain hydroxide ions in their chemical formula.

According to the present disclosure, the term “keratin fibers” means fibers of human or animal origin such as head hair, other body hairs, the eyelashes, wool, angora, cashmere or fur. Although the present disclosure is not limited to any category of keratin fibers, in at least one embodiment the present disclosure relates to head hair.

According to the present disclosure, the term “relaxing” includes the relaxing, smoothing out or straightening of Caucasian, Asiatic, North-African or African hair.

The term “polyguanidine not belonging to the hydroxide family,” as used herein, means any organic compound comprising in its formula at least 2 times the following group: a carbon atom doubly bonded to one other nitrogen atom and singly bonded to two other nitrogen atoms and containing no hydroxide ions in its chemical formula.

In at least one embodiment of the present disclosure, the polyguanidines may comprise the guanidine group at least 2 times, for example 2 or 3 times.

As used in the present disclosure, the expression “between x % and y %” means ranging from x to y %, the limits x and y being included.

Two techniques are used to permanently reshape the hair. They are based on breaking the disulphide bonds present in keratin (cystine):
the first technique comprises, in a first step, opening the disulphide bonds by means of a composition comprising a reducing agent, and then, after having optionally rinsed the hair, then in a second step, reconstituting the disulphide bonds by applying to the hair, which has been placed under tension beforehand with curlers or the like or shaped or smoothed out by other means, an oxidizing composition also known as a “fixing composition,” so as to give the head of hair the desired shape. This technique makes it possible alternatively to wave the hair or to relax it, straighten it or smooth it out.
The second technique comprises performing a lanthionization operation, using a composition containing a base belonging to the hydroxide family. This leads to replacement of the disulphide bonds (—CH₂—S—S—CH₂—) with lanthionine bonds (—CH₂—S—CH₂—). This lanthionization operation involves two consecutive chemical reactions:
The first reaction consists of a beta-elimination on the cystine, brought about by a hydroxide ion, leading to the breaking of this bond and the formation of dehydroalanine. 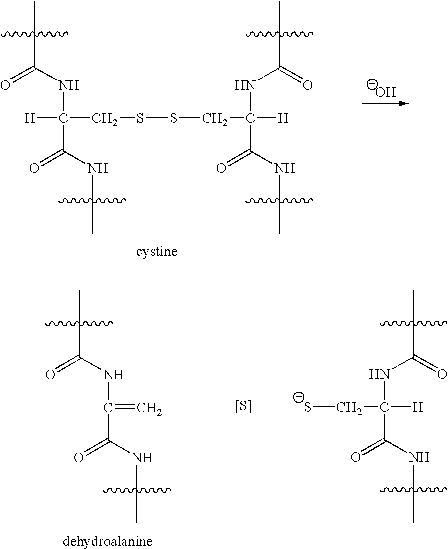
The second reaction is a reaction of the dehydroalanine with a thiol group. Specifically, the double bond of the dehydroalanine formed is a reactive double bond. It can react with the thiol group of the cysteine residue that has been released to form a new bond, referred to as a lanthionine bridge or bond or residue.

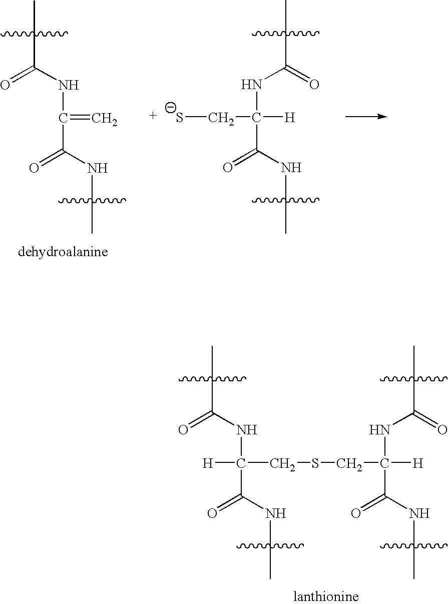

Relative to the first technique using a reducing agent, this lanthionization technique does not require a fixing step, since the formation of the lanthionine bridges is irreversible. It thus takes place in a single step and makes it possible either to wave the hair or to relax it, straighten it or smooth it out. However, it is generally used to relax naturally frizzy hair.

For the first technique, the reducing compositions generally used for the first step of a permanent-waving or relaxing operation contain thiols, sulphites or bisulphites as the reducing agent. These agents are typically used in essentially aqueous medium at concentrations ranging from 0.5M to 1 M to obtain good opening of the disulphide bonds. Among the thiols, those commonly used are thioglycolic acid, cysteamine, glyceryl monothioglycolate, thiolactic acid and cysteine. For example, thioglycolic acid is very efficient at reducing the disulphide bonds of keratin at alkaline pH, such as in the form of ammonium thioglycolate, and is the product most commonly used in permanent-waving (hair waving). However, it has been found that thioglycolic acid must be used in a sufficiently basic medium (in practice at a pH ranging from 8.5 to 9.5) if it is desired to obtain curling of sufficient intensity. Besides the drawback of releasing an unpleasant odor requiring the use of more or less efficient fragrances to mask the odors, the use of a thiol at alkaline pH may also lead to degradation of the fiber and possibly to impairment of the artificial colorations.

Sulphites or bisulphites are mainly used for relaxing. They have drawbacks similar to thiols, with lower efficacy.

Thiols and sulphites (or bisulphites) also have the drawback of having poor stability in aqueous solutions.

In general, the durability of the reshaping effects obtained with thiols and sulphites by reduction of disulphides followed by fixing is considered to be far inferior to that which may be obtained via the lanthionization technique.

For the second technique, the compositions typically used to perform the lanthionization contain a hydroxide as a base, such as sodium hydroxide, guanidinium hydroxide and lithium hydroxide. These lanthionization active agents, which make it possible to open the disulphide bonds via a beta-elimination mechanism, are typically used in water-oil emulsion at concentrations ranging from 0.4M to 0.6M, by leaving them to act for 10 to 15 minutes at room temperature. Sodium hydroxide is the agent most commonly used. Guanidinium hydroxide is the compound used in many compositions. These two hydroxides, sodium hydroxide and guanidinium hydroxide, are the two main agents used for relaxing or straightening naturally frizzy hair. They have several benefits over ammonium thioglycolate and sulphites, for example the absence of an unpleasant odor, the fact that only one implementation step is required (hence, shorter treatment time), and an improved durability and efficacy of the reshaping of the hair.

However, these hydroxides have the major drawback of typically being caustic. This causticity affects the scalp by causing irritation that on occasion is severe. This may be partially overcome by the prior application to the scalp of a greasy protective cream often referred to as a “base” or a “base cream”, the word “base” used here not having the meaning of a basic agent in the chemical sense. When the protective cream is combined with the hydroxide in a single composition, this is generally referred to as a “no-base” composition, as opposed to the above name. This “no-base” technology is used more often than the other.

The causticity of the hydroxides also affects the state of the hair by on the one hand giving it a coarse feel and on the other hand making it much more fragile, this fragility possibly going as far as fraying, breaking or even dissolution of the hair if the treatment is prolonged. In certain cases, hydroxides also cause decoloration of the natural color of the hair.

Formulations containing sodium hydroxide are generally referred to as “lye relaxers” and those not containing sodium hydroxide are known as “no-lye relaxers”.

The main “no-lye” relaxing formulations use guanidinium hydroxide. Since guanidinium hydroxide is unstable, it is generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate makes the final rinsing of the hair much more difficult and leaves on the hair and scalp mineral particles that give it a coarse feel and an unaesthetic appearance resembling dandruff. The recent success of guanidinium hydroxide (“no-lye”) over sodium hydroxide (“lye”) appears to arise from better relaxing efficacy and better skin tolerance. However, these technologies using bases of the hydroxide family remain very aggressive for the hair and the scalp and require very strict control of the application time to avoid excessive irritation and impairment of the hair that may go as far as breaking. This aggressiveness arising from the causticity of hydroxides is just reason for these compositions for the lanthionization of the hair not to be currently used for permanent-waving (hair waving), but to be reserved for relaxing (hair straightening or hair relaxing).

Furthermore, hydroxides are known to be good agents for hydrolysing amide functions (cf. for example, March's Advanced Organic Chemistry, 5th Edition, Wiley Interscience, New York, “Hydrolysis of Amides” pages 474 et seq.), which thus lead to breaking of the peptide bonds by direct nucleophilic attack. Thus, in the broad sense, it is probable that the impairments observed in the case of the hair and keratin materials are largely due to partial hydrolysis of the amide bonds of keratin.

Therefore, there is a real need, in hair relaxing, for compositions that are less aggressive to the hair and the skin.

Various studies have been performed with a view to simultaneously overcoming the drawbacks of reducing agents (first technique) and/or of hydroxides (second technique).

Thus, to replace thioglycolic acid, many reducing agents have been proposed, but thioglycolic acid in its ammonium thioglycolate form remains both the reference compound and the compound most widely used in cosmetic formulations, both for permanently shaping the hair and for relaxing it and smoothing it out.

In order to replace sodium hydroxide or guanidinium hydroxide and in order to improve skin tolerance, U.S. Pat. No. 4,530,830 has proposed using a composition based on quaternary ammonium hydroxides. However, these compositions have not always given full satisfaction, in terms either of relaxing or of cosmetology.

Many publications describe the use of hydroxides, serving as an active lanthionization agent, together with certain additives which serve generally to protect the hair.

For instance, without the use of new active lanthionization agents, the improvements proposed relate primarily to the use of additives for attenuating the damage caused to the hair by the hydroxides. They include, by way of non-limiting example:
Patent Application WO 2002/003937, which describes a composition containing C₃-C₅ monosaccharides,
Patent Application WO 2001/064171, which describes a composition containing complexing agents,
U.S. Pat. No. 5,641,477, which describes a composition containing a hydrogenated starch hydrolysate,
Patent Application WO 02/085317, which describes a composition containing organic nucleophiles, which react, during the second step, with the dehydroalanine formed with hydroxides, leading to new bridges,
U.S. Pat. No. 5,679,327, which describes a straightening composition mandatorily containing three active relaxing constituents, namely an alkali metal hydroxide, an alkaline-earth metal hydroxide, and a nitrogen-containing organic base, each of the constituents being present in the composition in a proportion which would be inadequate to bring about relaxing if it were used in the same concentration without the other two active agents. In other words, this patent describes a synergy between three constituents that in combination lead to the relaxing of keratin fibers.

Although all of these proposals lead to more or less significant improvements, they do not make it possible to sufficiently and consistently attenuate the harm associated with the actual causticity of the hydroxides.

In relation to the use of hydroxides for hair relaxing, U.S. Pat. No. 4,524,787 has disclosed, additionally, a ready-to-use composition produced from an “activator” portion, which is almost anhydrous and contains an organic base in a polyhydroxy alkane solvent, and an aqueous portion, which contains at least 20% of water. Nevertheless, this latter type of composition still does not always provide satisfaction, either in terms of quality of relaxing or in terms of mechanical and cosmetic properties of hair which has undergone this treatment.

As indicated above, the use of reducing agents may lead to mediocre durability for the relaxing or straightening, and the use of hydroxides, owing to their causticity, is limited in the hair relaxation field.

After extensive studies, it has now been surprisingly and unexpectedly discovered that the first step of the lanthionization process can be performed with polyguanidines not belonging to the hydroxide family. Excellent results in terms of hair relaxing, cosmetic and mechanical qualities of the hair can be thus obtained.

The present disclosure provides a cosmetic composition comprising, in a cosmetically acceptable medium, at least one polyguanidine not belonging to the hydroxide family, the cosmetically acceptable medium and the polyguanidine being chosen such that the polyguanidine not belonging to the hydroxide family reacts on the cystines of the keratin fibers, via a beta-elimination reaction, producing dehydroalanine and leading to the formation of lanthionine, to relax the keratin fibers in less than 60 minutes.

According to at least one embodiment, the relaxing time is less than 40 minutes, for instance less than 30 minutes.

As used herein, the expression “polyguanidines not belonging to the hydroxide family”, which may be used as beta-elimination active agents resulting in lanthionization, means any organic compound containing in its formula the following group at least 2 times: a carbon atom doubly bonded to one other nitrogen atom and singly bonded to two other nitrogen atoms containing no hydroxide ions in its formula and capable of accepting a proton.

The polyguanidines according to the present disclosure may, in at least one embodiment, be chosen from compounds having the following formula (I): 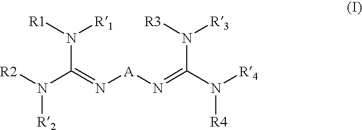
in which:
R1, R′₁, R2, R′₂, R3, R′₃, R4 and R′₄, which are identical or different, each are a radical chosen from:
   a hydrogen atom
   a saturated or unsaturated C₁ to C₆ alkyl group which is linear, such as methyl or ethyl, or branched, such as isopropyl or tert-butyl, or else cyclic, such as cyclopentyl or cyclohexyl, for example, the alkyl group being optionally substituted by the following radical: 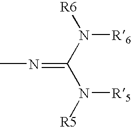
   in which R5, R′₅, R6 and R′₆ have the same meanings as the radicals R1 to R′₄ designated above.
R1 and R2 and/or R3 and R4 may also together form a divalent radical (CH₂)₂ or (CH₂)₃ or CH═CH.
A is a saturated or unsaturated, linear or cyclic divalent C₂ to C₁₂ hydrocarbon radical which may optionally be interrupted by at least one group such as imino, carboxamido, sulphoxide, sulphone, and/or by:
   at least one heteroatom chosen from sulphur, oxygen, nitrogen and silicon, and optionally substituted by:
   a linear or branched C₁ to C₄ alkyl radical optionally interrupted by at least one heteroatom as defined above and optionally substituted by the following radical: 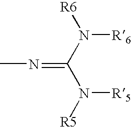
   in which R5, R′₅, R6 and R′₆ have the same meanings as the radicals R1 to R′₄ designated above.

The compounds of formula (I) may be prepared according to the following synthesis schemes:

Scheme 1:

The Vilsmeyer salt 1 (prepared by reacting phosgene or a substitute such as oxalyl chloride or phosphorous oxychloride with tetraalkylurea) is reacted with the diamine 2 (or triamine 3) in a polar aprotic solvent such as acetonitrile in the presence of a tertiary organic base, such as triethylamine, to give the compounds of formula (I). 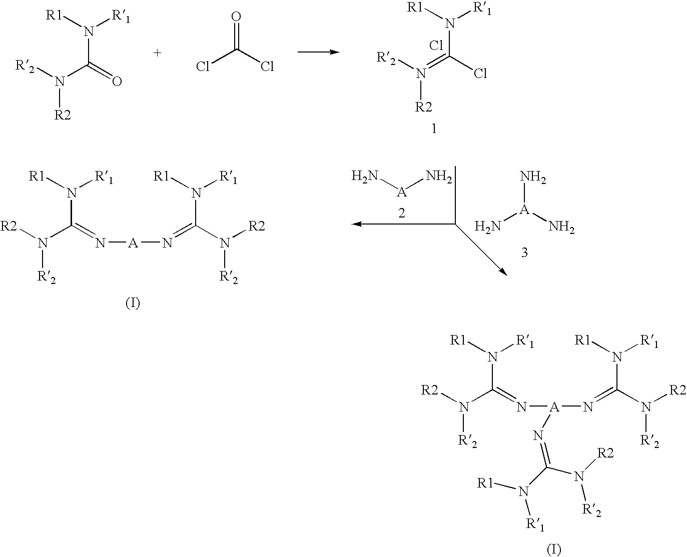
Scheme 2:

The dihalo derivative 4 is reacted with a large excess of the tetraalkylguanidine at a temperature in the region of 100° C. The excess tetraalkylguanidine is removed by evaporation under reduced pressure and the residue is treated with a base, such as sodium ethoxide in ethanol.

The salt is removed by filtration and then the expected derivative of formula (I) is obtained and is purified by distillation under reduced pressure.

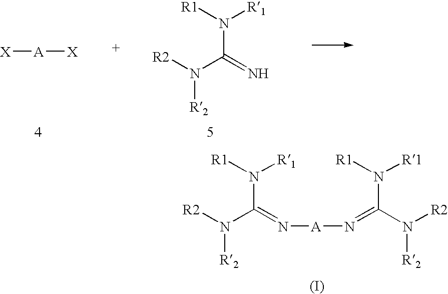

Reference may usefully be made to the procedures described in the following literature references:
1) Z. anorg. Allg. Chem. (2000), 626, pp. 1583-1590
2) Inorg. Chem. (2001), 40, pp. 6964-6971
3) J. Org. Chem. (2003), 68, pp. 8790-8797
4) J. Chem. Soc.; Dalton Trans., (2000), pp. 3473-3479

The compounds of formula (I) useful herein include but are not limited to:
—N,N′-bis(1,3-dimethyl-2-imidazolidinylidene)-1,2-benzenediamine (RN: 774610-65-0)
—N-[bis(dimethylamino)methylene]-3-(dimethylamino)-5-imino-2-methyl-2,4,6,19-tetraazaeicosane-20-imidamide (RN: 791543-84-5)
—N,N′-bis(tetrahydro-1,3-dimethyl-2(1H)-pyrimidinylidene)-1,3-propanediamine (RN: 752232-69-2)
-1,1′,1″,1tetrakis[1-propanediylbis(nitrilomethanetetrayl)piperidine (RN: 752232-68-1)
-poly(oxy-1,2-ethanediyl), α-[2-[(1,3-dimethyl-2-imidazolidinylidene)amino]ethyl]-ω-[2-[(1,3-dimethyl-2-imidazolidinylidene)amino]ethoxy] (RN: 742679-23-8)
—N″,N′-1,2-ethanediylbis[N,N,N′,N′-tetrakis(1-methylethyl)guanidine (RN: 676488-06-5)
—N,N′-bis(1,3-dihydro-1,3-dimethyl-2H-imidazol-2-ylidene)-1,8-naphthalenediamine (RN: 634192-99-7)
—N,N′-bis(1,3-dimethyl-2-imidazolidinylidene)-1,8-naphthalenediamine (RN: 501931-38-0)
—N″,N′-1,8-biphenylenediylbis[N,N,N′,N′-tetramethylguanidine] (RN: 495408-66-7)
—N″,N′-benzo[c]phenanthrene-1,12-diylbis[N,N,N′,N′-tetramethylguanidine] (RN: 495408-58-7)
—N″,N′-4,5-phenanthrenediylbis[N,N,N′,N′-tetramethylguanidine] (RN: 495408-50-9)
—N″,N′-(9,10-dihydro-4,5-phenanthrenediyl)bis[N,N,N′,N′-tetramethylguanidine] (RN: 495408-44-1)
—N″,N′-1,2-phenylenebis[N,N,N′,N′-tetramethylguanidine] (RN: 495408-35-0)
—N″,N′-1,8-naphthalenediylbis[N,N,N′,N′-tetramethylguanidine]hydrochloride (RN: 443892-20-4)
—N″,N′-9H-fluorene-4,5-diylbis[N,N,N′,N′-tetramethylguanidine] (RN: 443892-12-4)
—N′-(tetrahydro-1,3-dimethyl-2(1H)-pyrimidinylidene)-N,N-bis[2-[(tetrahydro-1,3-dimethyl-2(1H)-pyrimidinylidene)amino]ethyl]-1,2-ethanediamine (RN: 395640-62-7)
—N″,N′,N″″″″-(nitrilotri-2,1-ethanediyl)tris[N,N,N′,N′-tetramethylguanidine] (RN: 368866-05-1)
—N″, N′-[2-[[[bis(dimethylamino)methylene]amino]methyl]-2-methyl-1,3-propane-diyl]bis[N,N,N′,N′-tetramethylguanidine] (RN: 289474-30-2)
—N″,N′-(2,2-dimethyl-1-3-propanediyl)bis[N,N,N′,N′-tetramethylguanidine] (RN: 289474-29-9)
—N″,N′-1,3-propanediylbis[N,N,N′,N′-tetramethylguanidine] (RN: 289474-28-8)
—N″,N′-1,2-ethanediylbis[N,N,N′,N′-tetramethylguanidine] (RN: 289474-25-5)
—N,N′-bis(1,3-dihydro-1,3-dimethyl-2H-imidazol-2-ylidene)-1,2-ethanediamine (RN: 216873-26-6)
—N,N″-1,2-ethanediylbis[N′,N′,N″,N″-diethylguanidine] (RN: 211869-99-7)
—N″,N′-[(methylimino)di-3,1-propanediyl]bis[N,N,N′,N′-tetramethylguanidine] (RN: 196405-86-4)
—N″,N″-[oxybis(2,1-ethanediyloxy-3,1-propanediyl)]bis[N,N,N′,N′-tetramethylguanidine] (RN: 190442-53-6)
—N″,N″-[[1,1,3,3-tetramethyl-1,3-disiloxanediyl]di-3,1-propanediyl]bis[N,N,N′,N′-tetramethyl-guanidine] (RN: 175989-14-7)
—N″,N″-[[1,3-dimethyl-1,3-bis[(trimethylsilyl)oxy]-1,3-disiloxanediyl]di-3,1-propanediyl]bis[N,N,N′,N′-tetramethylguanidine] (RN: 172283-48-6)
—N,N″-1,6-hexanediylbis[N′-[bis(dimethylamino)methylene]urea] (RN: 157362-45-3)
-[bis(dimethylamino)methylene][3-[[[[[bis(dimethyl-amino)methylene]amino]carbonyl]amino]methyl]-3,5,5-trimethylcyclohexyl]urea (RN: 157362-44-2)
—N″,N″-[1,1′-biphenyl]-2,2′-diylbis[N,N,N′,N′-tetramethylguanidine] (RN: 144576-63-6)
-4,4′-sulphonylbis[N-(1,3-dimethyl-2-imidazolidinylidene)benzeneamine (RN: 129346-76-5)
—N,N′-bis(1,3-dimethyl-2-imidazolidinylidene)-2,8-dibenzothiophenediamine-5,5-dioxide (RN: 128169-35-7)
—N,N′-bis(1,3-dimethyl-2-imidazolidinylidene-4,6-dibenzothiophenediamine-5,5-dioxide (RN: 127330-56-7)
—N,N′-bis(1,3-dimethyl-2-imidazolidinylidene)-1,2-ethanediamine (RN: 126620-51-7)
—N″,N′-1,3-phenylenebis[N,N,N′,N′-tetramethylguanidine] (RN: 121648-84-8)
—N,N′-bis[bis(dimethylamino)methylene]butanediamine (RN: 114491-72-4)
—N″,N″-1,4-phenylenebis[N,N,N′,N′-tetramethylguanidine] (RN: 113551-45-4)
—N″,N″-[[1,3-dimethyl-1-[(pentamethyldisiloxanyl)oxy]-3-[(trimethylsilyl)oxy]-1,3-disiloxane-diyl]di-3,1-propanediyl]bis[N,N,N′,N′-tetramethylguanidine] (RN: 109956-31-2)
—N,N″-bis[(dimethylamino)(dipentylamino)methylene]-1,4-piperazinedicarboximidamide (RN: 97983-93-2)
—N,N″-bis[(dimethylamino)(hexylpropylamino)methylene]-1,4-piperazinedicarboximidamide (RN: 97983-92-1)
—N,N″-bis[(hexylmethylamino)(methyl propylamino)methylene)-1,4-piperazinedicarboximidamide (RN: 97983-91-0)
—N,N″-bis[(butylmethylamino)(hexylmethylamino)methylene)-1,4-piperazinedicarboximidamide (RN: 97983-90-9)
—N,N″-bis[(dimethylamino)(heptylmethylamino)methylene]-1,4-piperazinedicarboximidamide (RN: 97963-91-2)
—N″,N′-[(1,1,3,3-tetramethoxy-1,3-disiloxanediyl)di-3,1-propanediyl]bis[N,N,N′,N′-tetramethyl-guanidine] (RN: 69755-28-8)
—N″,N′-1,6-hexanediylbis[N,N,N′,N′-tetramethylguanidine] (RN: 64933-93-3)
—N″,N′-(methylenedi-4,1-phenylene)bis[N,N,N′,N′-tetramethylguanidine] (RN: 57414-23-0)
—N,N′-(methylphenylene)bis[N′-[bis(dimethylamino)methylene]urea] (RN: 39529-23-2)
-2,2′-(sulphonyldiethylene)bis[1,1,3,3-tetramethylguanidine] (RN: 13998-89-5)

In at least one embodiment, the compounds of formula (I) are chosen from:
—N-[bis(dimethylamino)methylene]-3-(dimethylamino)-5-imino-2-methyl-2,4,6,19-tetraazaeicosane-20-imidamide (RN: 791543-84-5)
—N,N′-bis(tetrahydro-1,3-dimethyl-2(1H)-pyrimidinylidene)-1,3-propanediamine (RN: 752232-69-2)
-poly(oxy-1,2-ethanediyl), α-[2-[(1,3-dimethyl-2-imidazolidinylidene)amino]ethyl]-ω-[2-[(1,3-dimethyl-2-imidazolidinylidene)amino]ethoxy] (RN: 742679-23-8)
—N″,N′-1,2-ethanediylbis[N,N,N′,N′-tetrakis(1-methylethyl)guanidine (RN: 676488-06-5)
—N,N′-bis(1,3-dihydro-1,3-dimethyl-2H-imidazol-2-ylidene)-1,8-naphthalenediamine (RN: 634192-99-7)
—N,N′-bis(1,3-dimethyl-2-imidazolidinylidene)-1,8-naphthalenediamine (RN: 501931-38-0)
—N″,N″-1,2-phenylenebis[N,N,N′,N′-tetramethylguanidine] (RN: 495408-35-0)
—N″,N″-1,8-naphthalenediylbis[N,N,N′,N′-tetramethylguanidine]hydrochloride (RN: 443892-20-4)
—N″,N″N″″″″-(nitrilotri-2,1-ethanediyl)tris[N,N,N′,N′-tetramethylguanidine] (RN: 368866-05-1)
—N″,N″-[2-[[[bis(dimethylamino)methylene]amino]methyl]-2-methyl-1,3-propanediyl]bis[N,N,N′,N′-tetramethylguanidine] (RN: 289474-30-2)
—N″,N″-(2,2-dimethyl-1,3-propanediyl)bis[N,N,N′,N′-tetramethylguanidine] (RN: 289474-29-9)
—N″,N″-1,3-propanediylbis[N,N,N′,N′-tetramethylguanidine] (RN: 289474-28-8)
—N,N′-bis(1,3-dihydro-1,3-dimethyl-2H-imidazol-2-ylidene)-1,2-ethanediamine (RN: 216873-26-6)
—N,N′-1,2-ethanediylbis[N′,N′,N″,N″-diethylguanidine] (RN: 211869-99-7)
—N″,N′-[(methylimino)di-3,1-propanediyl]bis[N,N,N′,N′-tetramethylguanidine] (RN: 196405-86-4)
—N″,N″-[oxybis(2,1-ethanediyloxy-3,1-propanediyl)]bis[N,N,N′,N′-tetramethylguanidine] (RN: 190442-53-6)
—N″,N″-[[1,1,3,3-tetramethyl-1,3-disiloxanediyl]di-3,1-propanediyl]bis[N,N,N′,N′-tetramethyl-guanidine] (RN: 175989-14-7)
—N,N″-1,6-hexanediylbis[N′-[bis(dimethylamino)methylene]urea] (RN: 157362-45-3)
—N″,N′-[1,1′-biphenyl]-2,2′-diylbis[N,N,N′,N′-tetramethylguanidine] (RN: 144576-63-6)
—N,N′-bis(1,3-dimethyl-2-imidazolidinylidene)-1,2-ethanediamine (RN: 126620-51-7)
—N″,N″-1,4-phenylenebis[N,N,N′,N′-tetramethylguanidine] (RN: 113551-45-4)
—N″,N″[(1,1,3,3-tetramethoxy-1,3-disiloxanediyl)di-3,1-propanediyl]bis[N,N,N′,N′-tetramethylguanidine] (RN: 69755-28-8)
—N″,N″1,6-hexanediylbis[N,N,N′,N′-tetramethylguanidine] (RN: 64933-93-3)
-2,2′-(sulphonyldiethylene)bis[1,1,3,3-tetramethylguanidine] (RN: 13998-89-5)

The present disclosure also relates to the novel compounds of formula (II): 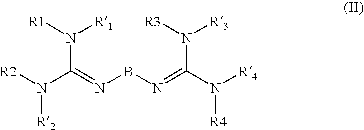
in which:
R1, R′₁, R2, R′₂, R3, R′₃, R4 and R′₄, which are identical or different, each are a group chosen from:
   a hydrogen atom
   a methyl, ethyl, propyl or isopropyl radical
R1 and R2 and/or R3 and R4 may also together form a divalent radical (CH₂)₂ or (CH₂)₃ or CH═CH.
B is a radical chosen from: 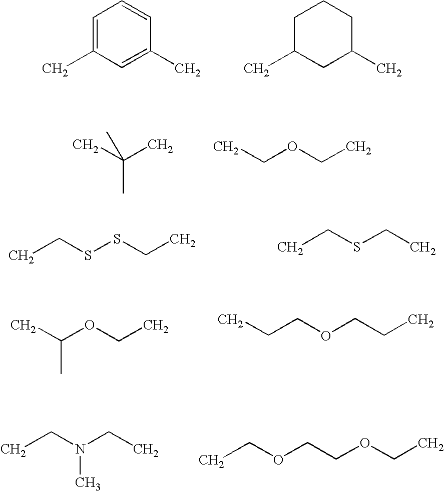
when R′₁═R′₂═R′₃═R′₄═CH₃ and when R1/R2 and R3/R4 respectively and simultaneously form the divalent radical (CH₂)₂:
B is a divalent radical (CH₂)₂ or (CH₂)₅ or (CH₂)₆ or a radical: 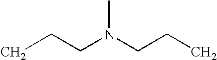
when R′₁═R′₂═R′₃═R′₄═CH₃ and when R1/R2 and R3/R4 respectively and simultaneously form the divalent radical (CH₂)₃:
B is (CH₂)₂ or (CH₂)₃ or (CH₂)₄ or (CH₂)₅ or (CH₂)₆ or a radical: 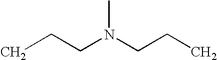
when R′₁═R′₂═R′₃═R′₄═CH₃ and when R1/R2 and R3/R4 respectively and simultaneously form the divalent radical CH═CH:
B is (CH₂)₃ or (CH₂)₄ or (CH₂)₅ or (CH₂)₆ or a radical: 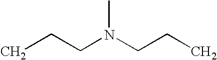
when R1, R′₁, R2, R′₂, R3, R′₃, R4 and R′₄ each are an ethyl or isopropyl radical, B is a (CH₂)₃ or (CH₂)₄ or (CH₂)₅ or (CH₂)₆ radical or a radical: 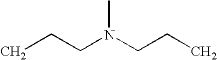
when R1, R′₁, R2, R′₂, R3, R′₃, R4 and R′₄ each are a methyl radical, B is a (CH₂)₄ or (CH₂)₅ radical.

The compounds of formula (II) useful herein include, but are not limited to:
N,N,N′,N′-tetraisopropyl-N″-[3-(N′,N′,N″,N″-tetraisopropylguanidino)propyl]guanidine
N,N,N′,N′-tetraisopropyl-N″-[4-(N′,N′,N″,N″-tetraisopropylguanidino)butyl]guanidine
N,N,N′,N′-tetraisopropyl-N″-[5-(N′,N′,N″,N″-tetraisopropylguanidino)pentyl]guanidine
N,N,N′,N′-tetraisopropyl-N″-[6-(N′,N′,N″,N″-tetraisopropylguanidino)hexyl]guanidine
N,N,N′,N′-tetraisopropyl-N″-(3-{methyl-[3-(N′,N′, N″,N″-tetraisopropylguanidino)propyl]amino}propyl)guanidine
N,N,N′,N′-tetraethyl-N″-[3-(N′,N′,N″,N″-tetraethylguanidino)propyl]guanidine
N,N,N′,N′,N′-tetraethyl-N″-[4-(N′,N′,N″,N″-tetraethylguanidino)butyl]guanidine
N,N,N′,N′-tetraethyl-N″-[5-(N′,N′,N″,N″-tetraethylguanidino)pentyl]guanidine
N,N,N′,N′-tetraethyl-N″-[6-(N′,N′,N″,N″-tetraethylguanidino)hexyl]guanidine
N,N,N′,N′-tetraethyl-N″-(3-{methyl-[3-(N′,N′,N″,N″-tetraisopropyl-guanidino)propyl]amino}propyl)guanidine
N,N′-bis(1,3-dimethylimidazolidin-2-ylidene)butane-1,4-diamine
N,N′-bis(1,3-dimethylimidazolidin-2-ylidene)pentane-1,5-diamine
N,N′-bis(1,3-dimethylimidazolidin-2-ylidene)hexane-1,6-diamine
N-(1,3-dimethylimidazolidin-2-ylidene)-N′-[3-(1,3-dimethylimidazolidin-2-ylideneamino)propyl]-N′-methylpropane-1,3-diamine
N,N′-bis(1,3-dimethyl-1,3-dihydroimidazol-2-ylidene)propane-1,3-diamine
N,N′-bis(1,3-dimethyl-1,3-dihydroimidazol-2-ylidene)butane-1,4-diamine
N,N′-bis(1,3-dimethyl-1,3-dihydroimidazol-2-ylidene)pentane-1,5-diamine
N,N′-bis(1,3-dimethyl-1,3-dihydroimidazol-2-ylidene)hexane-1,6-diamine
N-(1,3-dimethyl-1,3-dihydroimidazol-2-ylidene)-N′-[3-(1,3-dimethyl-1,3-dihydroimidazol-2-ylideneamino)propyl]-N′-methylpropane-1,3-diamine
N,N′-bis(1,3-dimethyltetrahydropyrimidin-2-ylidene)ethane-1,2-diamine
N,N′-bis(1,3-dimethyltetrahydropyrimidin-2-ylidene)propane-1,3-diamine
N,N′-bis(1,3-dimethyltetrahydropyrimidin-2-ylidene)butane-1,4-diamine
N,N′-bis(1,3-dimethyltetrahydropyrimidin-2-ylidene)pentane-1,5-diamine
N,N′-bis(1,3-dimethyltetrahydropyrimidin-2-ylidene)hexane-1,6-diamine
N-(1,3-dimethyltetrahydropyrimidin-2-ylidene)-N′-[3-(1,3-dimethyltetrahydropyrimidin-2-ylideneamino)propyl]-N′-methylpropane-1,3-diamine
N,N,N′,N′-tetramethyl-N″-[4-(N′,N′,N″,N″-tetramethylguanidino)butyl]guanidine
N,N,N′,N′-tetramethyl-N″-[5-(N′,N′,N″,N″-tetramethylguanidino)pentyl]guanidine
N,N,N′,N′-tetramethyl-N″-[3-(N′,N′,N″,N″-tetra-methylguanidinomethyl)cyclohexylmethyl]guanidine
N,N, N′,N′-tetraethyl-N″-[3-(N′,N′, N″,N″-tetra-ethylguanidinomethyl)cyclohexylmethyl]guanidine
N,N,N′,N′-tetrapropyl-N″-[3-(N′,N′, N″,N″-tetra-propylguanidinomethyl)cyclohexylmethyl]guanidine
N,N, N′,N′-tetraisopropyl-N″-[3-(N′,N′, N″,N″-tetra-isopropylguanidinomethyl)cyclohexylmethyl]guanidine
N,N,N′,N′-tetraethyl-N″-[3-(N′,N′,N″,N″-tetraethylguanidinomethyl)benzyl]guanidine
N,N,N′,N′-tetrapropyl-N″-[3-(N′,N′,N″,N″-tetrapropylguanidinomethyl)benzyl]guanidine
N,N,N′,N′-tetraisopropyl-N″-[3-(N′,N′,N″,N″-tetra-isopropylguanidinomethyl)benzyl]guanidine
N″-[2,2-dimethyl-3-(N′,N′,N″,N″-tetraethylguanidino)propyl]-N,N,N′,N′-tetraethylguanidine
N″-[2,2-dimethyl-3-(N′,N′,N″,N″-tetrapropylguanidino)propyl]-N,N,N′,N′-tetrapropylguanidine
N″-[2,2-dimethyl-3-(N′,N′,N″,N″-tetraisopropylguanidino)propyl]-N,N,N′,N′-tetraisopropylguanidine
N,N,N′,N′-tetramethyl-N″-{2-[2-(N′,N′,N″,N″-tetra-methylguanidino)ethoxy]ethyl}guanidine
N,N,N′,N′-tetraethyl-N″-{2-[2-(N′,N′,N″,N″-tetraethylguanidino)ethoxy]ethyl}guanidine
N N,N,N′,N′-tetrapropyl-N″-{2-[2-(N′,N′,N″,N″-tetra-propylguanidino)ethoxy]ethyl}guanidine
N,N,N′,N′-tetraisopropyl-N″-{2-[2-(N′,N′,N″,N″-tetra-isopropylguanidino)ethoxy]ethyl}guanidine
N,N,N′,N′-tetramethyl-N″-{2-[2-(N′,N′,N″,N″-tetra-methylguanidino)ethylsulphanyl]ethyl}guanidine
N,N,N′,N′-tetraethyl-N″-{2-[2-(N′,N′,N″,N″-tetra-ethylguanidino)ethylsulphanyl]ethyl}guanidine
N,N,N′,N′-tetrapropyl-N″-{2-[2-(N′,N′,N″,N″-tetra-propylguanidino)ethylsulphanyl]ethyl}guanidine
N,N,N′,N′-tetraisopropyl-N″-{2-[2-(N′,N′,N″,N″-tetraisopropylguanidino)ethylsulphanyl]ethyl}guanidine
N,N,N′,N′-tetramethyl-N″-{2-[2-(N′,N′, N″,N″-tetra-methylguanidino)ethyldisulphanyl]ethyl}guanidine
N,N,N′,N′-tetraethyl-N″-{2-[2-(N′,N′,N″, N″-tetra-ethylguanidino)ethyldisulphanyl]ethyl}guanidine
N,N,N′,N′-tetrapropyl-N″-{2-[2-(N′,N′,N″,N″-tetra-propylguanidino)ethyldisulphanyl]ethyl}guanidine
N,N,N′,N′-tetraisopropyl-N″-{2-[2-(N′,N′,N″,N″-tetraisopropylguanidino)ethyldisulphanyl]ethyl}guanidine
N,N,N′,N′-tetramethyl-N″-[2-methyl-5-(N′,N′,N″,N″-tetramethylguanidino)pentyl]guanidine
N,N,N′,N′-tetraethyl-N″-[2-methyl-5-(N′,N′,N″,N″-tetraethylguanidino)pentyl]guanidine
N,N,N′,N′-tetrapropyl-N″-[2-methyl-5-(N′,N′,N″, N″-tetrapropylguanidino)pentyl]guanidine
N,N,N′,N′-tetraisopropyl-N″-[2-methyl-5-(N′,N′,N″,N″-tetraisopropylguanidino)pentyl]-guanidine
N,N,N′,N′-tetramethyl-N″-{3-[3-(N′,N′,N″,N″-tetramethylguanidino)propoxy]propyl}guanidine
N,N,N′,N′-tetraethyl-N″-{3-[3-(N′,N′,N″,N″tetraethylguanidino)propoxy]propyl}guanidine
N,N,N′,N′-tetrapropyl-N″-{3-[3-(N′,N′,N″,N″-tetrapropylguanidino)propoxy]propyl}guanidine
N,N,N′,N′-tetraisopropyl-N″-{3-[3-(N′,N′,N″,N″-tetraisopropylguanidino)propoxy]propyl}guanidine
N,N,N′,N′-tetramethyl-N″-(2-{methyl-[2-(N′,N′,N″,N″-tetramethylguanidino)-ethyl]amino}ethyl)guanidine
N,N,N′,N′-tetraethyl-N″-(2-{methyl-[2-(N′,N′,N″,N″-tetraethylguanidino)ethyl]amino}-ethyl)guanidine
N,N,N′,N′-tetrapropyl-N″-(2-{methyl-[2-(N′,N′,N″,N″-tetrapropylguanidino)-ethyl]amino}ethyl)guanidine
N,N,N′,N′-tetraisopropyl-N″-(2-{methyl-[2-(N′,N′,N″,N″-tetraisopropylguanidino)-ethyl]amino}ethyl)guanidine
N,N,N′,N′-tetramethyl-N″-(2-{2-[2-(N′,N′,N″,N″-tetramethylguanidino)-ethoxy]ethoxy}ethyl)guanidine
N,N,N′,N′-tetraethyl-N″-(2-{2-[2-(N′,N′,N″,N″-tetraethylguanidino)-ethoxy]ethoxy}ethyl)guanidine
N,N,N′,N′-tetrapropyl-N″-(2-{2-[2-(N′,N′,N″,N″-tetrapropylguanidino)-ethoxy]ethoxy}ethyl)guanidine
N,N,N′,N′-tetraisopropyl-N″-(2-{2-[2-(N′,N′,N″,N″-tetraisopropylguanidino)-ethoxy]ethoxy}ethyl)guanidine
2-{3-[(1,3-dimethylimidazolidin-2-ylideneamino)methyl]cyclohexylmethylimino}-1,3-dimethylimidazolidine
2-{3-[(1,3-dimethylimidazolidin-2-ylideneamino)methyl]benzylimino}-1,3-dimethylimidazolidine
N,N′-bis(1,3-dimethylimidazolidin-2-ylidene)-2,2-dimethylpropane-1,3-diamine
2-{2-[2-(1,3-dimethylimidazolidin-2-ylideneamino)ethylsuphanyl]ethylimino}-1,3-dimethylimidazolidine
2-{2-[2-(1,3-dimethylimidazolidin-2-ylideneamino)ethyldisulphanyl]ethylimino}-1,3-dimethylimidazolidine
N1,N5-bis(1,3-dimethylimidazolidin-2-ylidene)-2-methylpentane-1,5-diamine
N-(1,3-dimethylimidazolidin-2-ylidene)-N′-[2-(1,3-dimethylimidazolidin-2-ylideneamino)ethyl]-N′-methylethane-1,2-diamine
N1,N5-bis(1,3-dimethyl-1,3-dihydroimidazol-2-ylidene)-2-methylpentane-1,5-diamine
N-(1,3-dimethyl-1,3-dihydroimidazol-2-ylidene)-N′-[2-(1,3-dimethyl-1,3-dihydroimidazol-2-ylideneamino)ethyl]-N′-methylethane-1,2-diamine
2-{3-[(1,3-dimethyl-1,3-dihydroimidazol-2-ylidene-amino)methyl]cyclohexylmethylimino}-1,3-dimethyl-2,3-dihydro-1H-imidazole
N,N′-bis(1,3-dimethyl-1,3-dihydroimidazol-2-ylidene)-2,2-dimethylpropane-1,3-diamine
2-{3-[(1,3-dimethyl-1,3-dihydroimidazol-2-ylideneamino)methyl]benzylimino}-1,3-dimethyl-2,3-dihydro-1H-imidazole
2-{2-[2-(1,3-dimethyl-1,3-dihydroimidazol-2-ylidene-amino)ethyldisulphanyl]ethylimino}-1,3-dimethyl-2,3-dihydro-1H-imidazole
2-{2-[2-(1,3-dimethyl-1,3-dihydroimidazol-2-ylideneamino)ethylsulphanyl]ethylimino}-1,3-dimethyl-2,3-dihydro-1H-imidazole
N1,N5-bis(1,3-dimethyltetrahydropyrimidin-2-ylidene)-2-methylpentane-1,5-diamine
N-1,3-dimethyltetrahydropyrimidin-2-ylidene)-N′-[2-(1,3-dimethyltetrahydropyrimidin-2-ylideneamino)ethyl]-N′-methylethane-1,2-diamine
2-{3-[(1,3-dimethyltetrahydropyrimidin-2-ylidene-amino)methyl]cyclohexylmethylimino}-1,3-dimethylhexahydropyrimidine
N,N′-bis(1,3-dimethyltetrahydropyrimidin-2-ylidene)-2,2-dimethylpropane-1,3-diamine
2-{3-[(1,3-dimethyltetrahydropyrimidin-2-ylideneamino)methyl]benzylimino}-1,3-dimethylhexahydropyrimidine
2-{2-[2-(1,3-dimethyltetrahydropyrimidin-2-ylidene-amino)ethyldisulphanyl]ethylimino}-1,3-dimethylhexahydropyrimidine
2-{2-[2-(1,3-dimethyltetrahydropyrimidin-2-ylideneamino)ethylsulphanyl]ethylimino}-1,3-dimethylhexahydropyrimidine

In at least one embodiment, useful compounds of formula (II) include:
N,N,N′,N′-tetraisopropyl-N″-[3-(N′,N′,N″,N″-tetraisopropylguanidino)propyl]guanidine
N,N,N′,N′-tetraisopropyl-N″-[4-(N′,N′,N″,N″-tetraisopropylguanidino)butyl]guanidine
N,N,N′,N′-tetraethyl-N″-[3-(N′,N′,N″,N″-tetraethylguanidino)propyl]guanidine
N,N,N′,N′-tetraethyl-N″-[4-(N′,N′,N″,N″-tetraethylguanidino)butyl]guanidine
N,N′-bis(1,3-dimethylimidazolidin-2-ylidene)butane-1,4-diamine
N-(1,3-dimethylimidazolidin-2-ylidene)-N′-[3-(1,3-dimethylimidazolidin-2-ylideneamino)propyl]-N′-methylpropane-1,3-diamine
N,N′-bis(1,3-dimethyl-1,3-dihydroimidazol-2-ylidene)propane-1,3-diamine
N,N′-bis(1,3-dimethyl-1,3-dihydroimidazol-2-ylidene)butane-1,4-diamine
N-(1,3-dimethyl-1,3-dihydroimidazol-2-ylidene)-N′-[3-(1,3-dimethyl-1,3-dihydroimidazol-2-ylideneamino)propyl]-N′-methylpropane-1,3-diamine
N,N′-bis(1,3-dimethyltetrahydropyrimidin-2-ylidene)ethane-1,2-diamine
N,N′-bis(1,3-dimethyltetrahydropyrimidin-2-ylidene)propane-1,3-diamine
N,N,N′,N′-tetramethyl-N″-[4-(N′,N′,N″,N″-tetramethylguanidino)butyl]guanidine
N,N,N′,N′-tetramethyl-N″-[3-(N′,N′,N″,N″-tetra-methylguanidinomethyl)cyclohexylmethyl]guanidine
N,N,N′,N′-tetraethyl-N″-[3-(N′,N′,N″,N″-tetraethylguanidinomethyl)benzyl]guanidine
N″-[2,2-dimethyl-3-(N′,N′,N″,N″-tetraethylguanidino)propyl]-N,N,N′,N′-tetraethylguanidine
N,N,N′,N′-tetramethyl-N″-{2-[2-(N′,N′,N″,N″-tetra-methylguanidino)ethoxy]ethyl}guanidine
N,N,N′,N′-tetramethyl-N″-{2-[2-(N′,N′,N″,N″-tetra-methylguanidino)ethylsulphanyl]ethyl}guanidine
N,N,N′,N′-tetramethyl-N″-{2-[2-(N′,N′,N″,N″-tetra-methylguanidino)ethyldisulphanyl]ethyl}guanidine
N,N,N′,N′-tetramethyl-N″-[2-methyl-5-(N′,N′,N″, N″-tetramethylguanidino)pentyl]guanidine
N,N,N′,N′-tetramethyl-N″-{3-[3-(N′,N′,N″, N″-tetra-methylguanidino)propoxy]propyl}guanidine
N N,N,N′,N′-tetramethyl-N″-(2-{methyl-[2-(N′,N′,N″, N″-tetramethylguanidino)-ethyl]amino}ethyl)guanidine
N,N,N′,N′-tetramethyl-N″-(2-{2-[2-(N′,N′,N″,N″-tetramethylguanidino)ethoxy]ethoxy}ethyl)guanidine
2-{3-[(1,3-dimethylimidazolidin-2-ylideneamino)methyl]cyclohexylmethylimino}-1,3-dimethylimidazolidine
2-{2-[([2-(1,3-dimethylimidazolidin-2-ylideneamino)ethylsulphanyl]ethylimino}-1,3-dimethylimidazolidine
N-(1,3-dimethyl-1,3-dihydroimidazol-2-ylidene)-N′-[2-(1,3-dimethyl-1,3-dihydroimidazol-2-ylideneamino)ethyl]-N′-methylethane-1,2-diamine
2-{2-[2-(1,3-dimethyl-1,3-dihydroimidazol-2-ylidene-amino)ethyldisulphanyl]ethylimino}-1,3-dimethyl-2,3-dihydro-1H-imidazole
N1,N5-bis(1,3-dimethyltetrahydropyrimidin-2-ylidene)-2-methylpentane-1,5-diamine
2-{3-[(1,3-dimethyltetrahydropyrimidin-2-ylidene-amino)methyl]benzyl imino}-1,3-dimethylhexahydropyrimidine

The novel compounds of formula (II) can be prepared in accordance with the synthesis schemes described for the compounds of formula (I).

The present disclosure also relates to the use in cosmetology, for instance in hair cosmetology, such as in hair relaxing, of the compounds of formula (II), and also to a cosmetic process, for example a hair cosmetic process and further for example, a hair relaxing process, which uses at least one compound of formula (II).

The compounds of formula (II) can be used for any keratin material, such as for the hair and the skin, as a care, protection, makeup or form retention product. For example, they may be used in lipsticks, creams for the body or hands, eyeshadows, mascaras, eyeliners or blushes.

In the compositions according to the present disclosure intended for a process for relaxing, straightening or smoothing out the hair, the polyguanidine not belonging to the hydroxide family may, for instance, be present in a molar concentration ranging from 0.1 M to 2M, which corresponds to concentrations ranging from 1.4% to 80% by weight relative to the total weight of the composition, and further for example, in a concentration ranging from 0.2M to 1 M, which corresponds to concentrations ranging from 2.8% to 40% by weight relative to the total weight of the composition.

In the cosmetic compositions, for instance the hair relaxing compositions, the molar and weight concentrations of the compounds of formula (II) are the same as for the compounds of formula (I).

The pH of the compositions according to the present disclosure may range from 9.6 to 14, for example from 11 to 13.

According to at least one embodiment, in the compositions of the present disclosure, the polyguanidine not belonging to the hydroxide family is the only relaxing active agent.

The compositions according to the present disclosure may also contain known reducing agents, for instance thioglycolic acid or thiolactic acid and ester and amide derivatives thereof, such as glyceryl monothioglycolate, cysteamine and its C₁-C₄ acyl derivatives such as N-acetylcysteamine or N-propionylcysteamine, cysteine, N-acetylcysteine, thiomalic acid, pantetheine, 2,3-dimercaptosuccinic acid, sulphites or bisulphites of an alkali metal or alkaline-earth metal, the N-(mercaptoalkyl)-w-hydroxyalkylamides described in European Patent Application EP-A-354 835, the N-mono- or N,N-dialkylmercapto-4-butyramides described in European Patent Application EP-A-368 763, the aminomercaptoalkylamides described in European Patent Application EP-A-432 000, the N-(mercaptoalkyl)succinamic acid and N-(mercaptoalkyl)succinimide derivatives described in European Patent Application EP-A-465 342, the alkylamino mercaptoalkylamides described in European Patent Application EP-A-514 282, the azeotropic mixture of 2-hydroxypropyl thioglycolate and of (2-hydroxy-1-methyl)ethyl thioglycolate described in French Patent Application FR-A-2 679 448, the mercaptoalkyl-aminoamides described in French Patent Application FR-A-2 692 481, the N-mercaptoalkylalkanediamides described in European Patent Application EP-A-653 202 and the formamidinesulphinic acid derivatives described in PCT Patent Application PCT/US 01/43124.

When the compositions, according to the present disclosure, contain at least one reducing agent, this agent may be present in a maximum concentration of 20% by weight relative to the total weight of the composition, for example, and further, for example, ranging in concentration from 0.1% to 10% by weight relative to the total weight of the composition.

The compositions according to the present disclosure may also contain known hydroxides that can be chosen from alkali metal or alkaline-earth metal or transition metal or organic hydroxides such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and quaternary ammonium hydroxides.

When the compositions of the present disclosure contain at least one hydroxide, this hydroxide may be present in a concentration ranging from 0.01% to 3.5% by weight relative to the total weight of the composition, for instance, and further, for example ranging from 0.05% to 1.5% by weight relative to the total weight of the composition.

For instance, according to at least one embodiment of present disclosure, the compositions as disclosed contain 0% of base belonging to the hydroxide family, chosen for instance from alkali metal or alkaline-earth metal or transition metal or organic hydroxides.

The compositions of the present disclosure may contain from 0 to 50% of water, for example, from 0 to 30% and further, for example, from 0 to 20%.

According to at least one embodiment, the basic compositions also contain a surfactant of nonionic, anionic, cationic or amphoteric type, and among these agents mention may be made of alkyl sulphates, alkylbenzene sulphates, alkyl ether sulphates, alkyl sulphonates, quaternary ammonium salts, alkylbetaines, oxyethylenated alkylphenols, fatty acid alkanolamides, oxyethylenated fatty acid esters and other nonionic surfactants of the hydroxypropyl ether type.

When the basic compositions contain at least one surfactant, this surfactant is present in a maximum concentration of 30% by weight relative to the total weight of the composition, for instance ranging from 0.5% to 10% by weight relative to the total weight of the composition.

With the aim of improving the cosmetic properties of the hair or of attenuating or avoiding its degradation, the basic composition may also contain a treating agent of cationic, anionic, nonionic or amphoteric nature.

Among the treating agents that may be used, non-limiting mention may be made to those described in French Patents FR-2 598 613 and FR-2 470 596. It is also possible to use as treating agents volatile or non-volatile, linear or cyclic silicones and mixtures thereof, polydimethylsiloxanes, quaternized polyorganosiloxanes such as those described in French Patent Application FR-2 535 730, polyorganosiloxanes containing aminoalkyl groups modified with alkoxycarbonylalkyl groups, such as those described in U.S. Pat. No. 4,749,732, polyorganosiloxanes such as the polyoxyalkyl polydimethylsiloxane copolymer of the Dimethicone Copolyol type, a polydimethylsiloxane containing stearoxy end groups (stearoxy dimethicone), a dialkylammonium acetate polydimethylsiloxane copolymer or a polydimethylsiloxane polyalkylbetaine copolymer described in British Patent GB-2 197 352, polysiloxanes organomodified with mercapto or mercaptoalkyl groups, such as those described in French Patent FR-1 530 369 and in European Patent Application EP 0 295 780, and also silanes such as stearoxy-trimethylsilane.

The basic compositions according to the present disclosure may also contain other treating ingredients such as cationic polymers, for instance those used in the compositions of French Patents FR-79/32078 (FR-2 472 382) and FR-80/26421 (FR-2 495 931) or cationic polymers of the ionene type, such as those used in the compositions of Luxembourg Patent 83703, basic amino acids (such as lysine or arginine) or acidic amino acids (such as glutamic acid or aspartic acid), peptides and derivatives thereof, protein hydrolysates, waxes, swelling agents and penetrating agents or agents for reinforcing the efficacy of the reducing agent, such as the SiO₂/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers, for instance propylene glycol monomethyl ether, dipropylene glycol monomethyl ether, ethylene glycol monoethyl ether and diethylene glycol monoethyl ether, 2-imidazolidinone, and also other compounds such as fatty alcohols, lanolin derivatives, active ingredients such as pantothenic acid, agents for preventing hair loss, anti-dandruff agents, thickeners, suspending agents, sequestering or complexing agents, opacifiers, sunscreens, fragrances and preserving agents.

For instance, in the compositions according to the present disclosure, the polyguanidine not belonging to the hydroxide family relaxes keratin fibers without being placed in contact beforehand with an organic solvent.

The compositions according to the present disclosure are, in at least one embodiment, in the form of a thickened cream so as to hold the hair as stiff as possible. These creams are made in the form of “heavy” emulsions, for example based on glyceryl stearate, glycol stearate, self-emulsifying waxes or fatty alcohols.

Liquids or gels containing thickeners, such as carboxyvinyl polymers or copolymers that “stick” the hairs together and hold them in the smooth position during the leave-in time, may also be used.

The compositions according to the present disclosure may also contain at least one adjuvant chosen from silicones in soluble, dispersed or microdispersed form, nonionic, anionic, cationic and amphoteric surfactants, ceramides, glycoceramides and pseudoceramides, vitamins and provitamins including panthenol, plant, animal, mineral and synthetic oils, waxes other than ceramides, glycoceramides and pseudoceramides, water-soluble and liposoluble, silicone-based or non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents. Among the solubilizers, non-limiting mention may be made, for example, of lower alcohols, such as ethanol, propanol or isopropanol, for example.

The present disclosure also relates to a kit comprising at least two compartments, one of the compartments (i) comprising, in a cosmetically acceptable medium, at least one polyguanidine not belonging to the hydroxide family, which is capable of reacting with the cystines of keratin fibers, via a beta-elimination reaction producing dehydroalanine and leading to the formation of lanthionine, to relax the keratin fibers in less than 60 minutes.

In at least one embodiment, the kit according to the present disclosure also comprises an additional composition (ii) for caring for, conditioning, making up, removing makeup from, protecting, cleansing or washing keratin fibers.

The compositions of the kits according to the present disclosure are packaged in separate compartments, containers or devices, optionally accompanied by suitable, identical or different application means, such as fine brushes, coarse brushes or sponges.

Another aspect of the present disclosure concerns a process for relaxing keratin materials using a cosmetic composition comprising, in a cosmetically acceptable medium, at least one polyguanidine not belonging to the hydroxide family, the cosmetically acceptable medium and the polyguanidine being chosen such that the polyguanidine not belonging to the hydroxide family reacts on the cystines of the keratin fibers, via a beta-elimination reaction producing dehydroalanine and leading to the formation of lanthionine, to relax the keratin fibers in less than 60 minutes.

For instance, in the process according to the present disclosure, the relaxing time is less than 40 minutes, for example less than 30 minutes.

In the hair relaxing, straightening or smoothing process according to the present disclosure, the basic composition as disclosed is applied to the hair, and then the hair is subjected to mechanical deformation which allows it to be given a new form, by an operation of smoothing out the hair with a wide-toothed comb, with the back of a comb or with the hand. After a leave-in time of 5 to 60 minutes, for instance of 5 to 40 minutes, smoothing out is repeated, and then the hair is rinsed abundantly.

According to the present disclosure, after application of the disclosed composition, the head of hair may, in at least one embodiment, be subjected to a heat treatment by heating to a temperature ranging from 30 to 60° C. In practice, this operation may be performed using a hairstyling hood, a hairdryer, an infrared ray dispenser and other standard heating devices.

It is possible to use a heating iron at a temperature ranging from 60 to 220° C. and further ranging from 120 to 200° C. as a means of both heating and smoothing out the hair.

Yet another aspect of the present disclosure concerns the use of a polyguanidine not belonging to the hydroxide family as an active agent for relaxing keratin fibers.

The present disclosure also relates to an active agent for relaxing keratin fibers, by means of a beta-elimination reaction producing dehydroalanine and leading to the formation of lanthionine, comprising at least one polyguanidine not belonging to the hydroxide family.

The present disclosure may be understood more clearly with the aid of the non-limiting examples that follow, which constitute several embodiments of the compositions according to the present disclosure.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the illustrative examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLE 1

A simplified hair relaxing composition was prepared, containing N″,N″-1,3-propanediylbis[N,N,N′,N′-tetramethylguanidine] (RN: 289474-28-8) at a concentration of 0.5M in water, as active relaxing agent. This composition was applied to naturally frizzy African hair for 20 minutes at a temperature of 30° C. The hair was effectively relaxed, was easy to comb and to style, and felt soft.

### EXAMPLE 2

A simplified hair relaxing composition was prepared, containing N″,N″,N″″-(nitrilotri-2,1-ethanediyl)tris[N,N,N′,N′-tetramethylguanidine] (RN: 368866-05-1) at a concentration of 0.25M in water, as active relaxing agent. This composition was applied to naturally frizzy African hair for 25 minutes at a temperature of 30° C. The hair was effectively relaxed, was easy to comb and to style, and felt soft.

What is claimed is:

1. A cosmetic composition for relaxing keratin fibers, said composition comprising, in a cosmetically acceptable medium, at least one polyguanidine not belonging to the hydroxide family, the cosmetically acceptable medium and the polyguanidine being chosen such that the polyguanidine not belonging to the hydroxide family reacts on the cystines of keratin fibers, via a beta-elimination reaction, producing dehydroalanine and leading to the formation of lanthionine, to relax the keratin fibers in less than 60 minutes.

2. The cosmetic composition according to claim 1, wherein the relaxing time is less than 40 minutes.

3. The cosmetic composition according to claim 2, wherein the relaxing time is less than 30 minutes.

4. The cosmetic composition according to claim 1, wherein the polyguanidine not belonging to the hydroxide family is present in a molar concentration ranging from 0.1 M to 2M.

5. The cosmetic composition according to claim 4, wherein the polyguanidine not belonging to the hydroxide family is present in a molar concentration ranging from 0.2M and 1M.

6. The cosmetic composition according to claim 1, wherein the pH ranges from 9.6 to 14.

7. The cosmetic composition according to claim 6, wherein the pH ranges from 11 to 13.

8. The cosmetic composition according to claim 1, wherein the cosmetic composition contains 0% of base belonging to the hydroxide family.

9. The cosmetic composition according to claim 1, wherein the cosmetic composition contains an amount of water ranging from 0 to 50% relative to the total weight of the composition.

10. The cosmetic composition according to claim 1, wherein the cosmetic composition contains an amount of water ranging from 0 to 20% relative to the total weight of the composition.

11. The cosmetic composition according to claim 1, wherein the polyguanidine is chosen from compounds of the following formula (I): 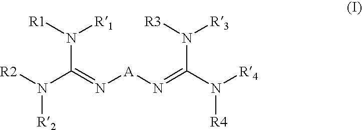
in which:
R1, R′₁, R2, R′₂, R3, R′₃, R4 and R′₄, which are identical or different, are each a radical chosen from:
a hydrogen atom
a saturated or unsaturated, linear, branched, or cyclic C₁ to C₆ alkyl group, the alkyl group being optionally substituted by the following radical: 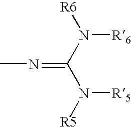
in which R5, R′₅, R6 and R′₆ have the same meanings as the radicals R1 to R′₄ defined above,
R1 and R2 and/or R3 and R4 may also together form a divalent radical chosen from (CH₂)₂, (CH₂)₃ and CH═CH,
A is a saturated or unsaturated, linear or cyclic divalent C₂ to C₁₂ hydrocarbon radical which may optionally be interrupted by at least one group chosen from imino, carboxamido, sulphoxide, and sulphone groups, and/or by at least one heteroatom chosen from sulphur, oxygen, nitrogen and silicon, and optionally substituted by a linear or branched C₁ to C₄ alkyl radical optionally interrupted by at least one heteroatom as defined above and optionally substituted by the following radical: 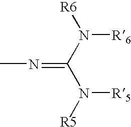
in which R5, R′₅, R6 and R′₆ have the same meanings as the radicals R1 to R′₄ defined above.

12. The cosmetic composition according to claim 1, wherein the compounds of formula (I) are chosen from:
—N,N′-bis(1,3-dimethyl-2-imidazolidinylidene)-1,2-benzenediamine (RN: 774610-65-0)
—N-[bis(dimethylamino)methylene]-3-(dimethylamino)-5-imino-2-methyl-2,4,6,19-tetraazaeicosane-20-imidamide (RN: 791543-84-5)
—N,N′-bis(tetrahydro-1,3-dimethyl-2(1H)-pyrimidinylidene)-1,3-propanediamine (RN: 752232-69-2)
-1,1′,1″,1-tetrakis[1-propanediylbis(nitrilo-methanetetrayl)]piperidine (RN: 752232-68-1)
-poly(oxy-1,2-ethanediyl), α-[2-[(1,3-dimethyl-2-imidazolidinylidene)amino]ethyl]-ω-[2-[(1,3-dimethyl-2-imidazolidinylidene)amino]ethoxy] (RN: 742679-23-8)
—N″,N′-1,2-ethanediylbis[N,N,N′,N′-tetrakis(1-methylethyl)guanidine (RN: 676488-06-5)
—N,N′-bis(1,3-dihydro-1,3-dimethyl-2H-imidazol-2-ylidene)-1,8-naphthalenediamine (RN: 634192-99-7)
—N,N′-bis(1,3-dimethyl-2-imidazolidinylidene)-1,8-naphthalenediamine (RN: 501931-38-0)
—N″,N′-1,8-biphenylenediylbis[N,N,N′,N′-tetramethylguanidine] (RN: 495408-66-7)
—N″,N′-benzo[c]phenanthrene-1,12-diylbis[N,N,N′,N′-tetramethylguanidine] (RN: 495408-58-7)
—N″,N′-4,5-phenanthrenediylbis[N,N,N′,N′-tetramethylguanidine] (RN: 495408-50-9)
—N″,N′-(9,10-dihydro-4,5-phenanthrenediyl)bis[N,N,N′,N′-tetramethylguanidine] (RN: 495408-44-1)
—N″,N′-1,2-phenylenebis[N,N,N′,N′-tetramethylguanidine] (RN: 495408-35-0)
—N″,N′-1,8-naphthalenediylbis[N,N,N′,N′-tetramethylguanidine]hydrochloride (RN: 443892-20-4)
—N″,N′-9H-fluorene-4,5-diylbis[N,N,N′,N′-tetramethylguanidine] (RN: 443892-12-4)
—N′-(tetrahydro-1,3-dimethyl-2(1H)-pyrimidinylidene)-N,N-bis[2-[(tetrahydro-1,3-dimethyl-2(1H)-pyrimidinylidene)amino]ethyl]-1,2-ethanediamine (RN: 395640-62-7)
—N″,N′,N″″″″-(nitrilotri-2,1-ethanediyl)tris[N,N,N′,N′-tetramethylguanidine] (RN: 368866-05-1)
—N″,N′-[2-[[[bis(dimethylamino)methylene]amino]methyl]-2-methyl-1,3-propane-diyl]bis[N,N,N′,N′-tetramethylguanidine] (RN: 289474-30-2)
—N″,N′-(2,2-dimethyl-1,3-propanediyl)bis[N,N,N′,N′-tetramethylguanidine] (RN: 289474-29-9)
—N″,N′-1,3-propanediylbis[N,N,N′,N′-tetramethylguanidine] (RN: 289474-28-8)
—N″,N′-1,2-ethanediylbis[N,N,N′,N′-tetramethylguanidine] (RN: 289474-25-5)
—N,N′-bis(1,3-dihydro-1,3-dimethyl-2H-imidazol-2-ylidene)-1,2-ethanediamine (RN: 216873-26-6)
—N,N′-1,2-ethanediylbis[N′,N′,N″,N″-diethylguanidine] (RN: 211869-99-7)
—N″,N′-[(methylimino)di-3,1-propanediyl]bis[N,N,N′,N′-tetramethylguanidine] (RN: 196405-86-4)
—N″,N″-[oxybis(2,1-ethanediyloxy-3,1-propanediyl)]bis[N,N,N′,N′-tetramethylguanidine] (RN: 190442-53-6)
—N″,N″-[[1,1,3,3-tetramethyl-1,3-disiloxanediyl]di-3,1-propanediyl]bis[N,N,N′,N′-tetramethylguanidine] (RN: 175989-14-7)
—N″,N″-[[1,3-dimethyl-1,3-bis[(trimethylsilyl)oxy]-1,3-disiloxanediyl]di-3,1-propanediyl]bis[N,N,N′,N′-tetramethylguanidine] (RN: 172283-48-6)
—N,N″-1,6-hexanediylbis[N′-[bis(dimethylamino)methylene]urea] (RN: 157362-45-3)
-[bis(dimethylamino)methylene][3-[[[[[bis(dimethylamino)methylene]amino]carbonyl]-amino]methyl]-3,5,5-trimethylcyclohexyl]urea (RN: 157362-44-2)
—N″,N″-[1,1′-biphenyl]-2,2′-diylbis[N,N,N′,N′-tetramethylguanidine] (RN: 144576-63-6)
-4,4′-sulphonylbis[N-(1,3-dimethyl-2-imidazolidinylidene)benzeneamine (RN: 129346-76-5)
—N,N′-bis(1,3-dimethyl-2-imidazolidinylidene)-2,8-dibenzothiophenediamine-5,5-dioxide (RN: 128169-35-7)
—N,N′-bis(1,3-dimethyl-2-imidazolidinylidene)-4,6-dibenzothiophenediamine-5,5-dioxide (RN: 127330-56-7)
—N,N′-bis(1,3-dimethyl-2-imidazolidinylidene)-1,2-ethanediamine (RN: 126620-51-7)
—N″,N′-1,3-phenylenebis[N,N,N′,N′-tetramethylguanidine] (RN: 121648-84-8)
—N,N′-bis[bis(dimethylamino)methylene]butanediamine (RN: 114491-72-4)
—N″,N″-1,4-phenylenebis[N,N,N′,N′-tetramethylguanidine] (RN: 113551-45-4)
—N″,N′-[[1,3-dimethyl-1-[(pentamethyldisiloxanyl)oxy]-3-[(trimethylsilyl)oxy]-1,3-disiloxanediyl]di-3,1-propanediyl]bis[N,N,N′,N′-tetramethylguanidine] (RN: 109956-31-2)
—N,N″-bis[(dimethylamino)(dipentylamino)methylene]-1,4-piperazinedicarboximidamide (RN: 97983-93-2)
—N,N″-bis[(dimethylamino)(hexylpropylamino)methylene]-1,4-piperazinedicarboximidamide (RN: 97983-92-1)
—N,N″-bis[(hexylmethylamino)(methylpropylamino)methylene)-1,4-piperazinedicarboximidamide (RN: 97983-91-0)
—N,N″-bis[(butylmethylamino)(hexylmethylamino)methylene)-1,4-piperazinedicarboximidamide (RN: 97983-90-9)
—N,N″-bis[(dimethylamino)(heptylmethylamino)methylene]-1,4-piperazinedicarboximidamide (RN: 97963-91-2)
—N″,N′-[(1,1,3,3-tetramethoxy-1,3-disiloxanediyl)di-3,1-propanediyl]bis[N,N,N′,N′-tetramethylguanidine] (RN: 69755-28-8)
—N″,N′-1,6-hexanediylbis[N,N,N′,N′-tetramethylguanidine] (RN: 64933-93-3)
—N″,N′-(methylenedi-4,1-phenylene)bis[N,N,N′,N′-tetramethylguanidine] (RN: 57414-23-0)
—N,N″-(methylphenylene)bis[N′-[bis(dimethylamino)methylene]urea] (RN: 39529-23-2)
-2,2′-(sulphonyldiethylene)bis[1,1,3,3-tetramethylguanidine] (RN: 13998-89-5)

13. The cosmetic composition according to claim 12, wherein the compounds of formula (I) are chosen from:
—N-[bis(dimethylamino)methylene]-3-(dimethylamino)-5-imino-2-methyl-2,4,6,19-tetraazaeicosane-20-imidamide (RN: 791543-84-5)
—N,N′-bis(tetrahydro-1,3-dimethyl-2(1H)-pyrimidinylidene)-1,3-propanediamine (RN: 752232-69-2)
-poly(oxy-1,2-ethanediyl), α-[2-[(1,3-dimethyl-2-imidazolidinylidene)amino]ethyl]-ω-[2-[(1,3-dimethyl-2-imidazolidinylidene)amino]ethoxy] (RN: 742679-23-8)
—N″,N′-1,2-ethanediylbis[N,N,N′,N′-tetrakis(1-methylethyl)guanidine (RN: 676488-06-5)
—N,N′-bis(1,3-dihydro-1,3-dimethyl-2H-imidazol-2-ylidene)-1,8-naphthalenediamine (RN: 634192-99-7)
—N,N′-bis(1,3-dimethyl-2-imidazolidinylidene)-1,8-naphthalenediamine (RN: 501931-38-0)
—N″,N″-1,2-phenylenebis[N,N,N′,N′-tetramethylguanidine] (RN: 495408-35-0)
—N″,N″-1,8-naphthalenediylbis[N,N,N′,N′-tetramethylguanidine]hydrochloride (RN: 443892-20-4)
—N″,N″,N″″″″-(nitrilotri-2,1-ethanediyl)tris[N,N,N′,N′-tetramethylguanidine] (RN: 368866-05-1)
—N″,N″-[2-[[[bis(dimethylamino)methylene]amino]methyl]-2-methyl-1,3-propanediyl]bis[N,N,N′,N′-tetramethylguanidine] (RN: 289474-30-2)
—N″,N″-(2,2-dimethyl-1,3-propanediyl)bis[N,N,N′,N′-tetramethylguanidine] (RN: 289474-29-9)
—N″,N″-1,3-propanediylbis[N,N,N′,N′-tetramethylguanidine] (RN: 289474-28-8)
—N,N′-bis(1,3-dihydro-1,3-dimethyl-2H-imidazol-2-ylidene)-1,2-ethanediamine (RN: 216873-26-6)
—N,N′-1,2-ethanediylbis[N′,N′,N″,N″-diethylguanidine] (RN: 211869-99-7)
—N″,N′-[(methylimino)di-3,1-propanediyl]bis[N,N,N′,N′-tetramethylguanidine] (RN: 196405-86-4)
—N″,N″-[oxybis(2,1-ethanediyloxy-3,1-propanediyl)]bis[N,N,N′,N′-tetramethylguanidine] (RN: 190442-53-6)
—N″,N″-[[1,1,3,3-tetramethyl-1,3-disiloxanediyl]di-3,1-propanediyl]bis[N,N,N′,N′-tetramethylguanidine (RN: 175989-14-7)
—N,N″-1,6-hexanediylbis[N′-[bis(dimethylamino)methylene]urea] (RN: 157362-45-3)
—N″,N″-[1,1′-biphenyl]-2,2′-diylbis[N,N,N′,N′-tetramethylguanidine] (RN: 144576-63-6)
—N,N′-bis(1,3-dimethyl-2-imidazolidinylidene)-1,2-ethanediamine (RN: 126620-51-7)
—N″,N″-1,4-phenylenebis[N,N,N′,N′-tetramethylguanidine] (RN: 113551-45-4)
—N″,N′-[(1,1,3,3-tetramethoxy-1,3-disiloxanediyl)di-3,1-propanediyl]bis[N,N,N′,N′-tetramethylguanidine] (RN: 69755-28-8)
—N″,N″-1,6-hexanediylbis[N,N,N′,N′-tetramethylguanidine] (RN: 64933-93-3)
-2,2′-(sulphonyldiethylene)bis[1,1,3,3-tetramethylguanidine] (RN: 13998-89-5)

14. A compound of formula (II): 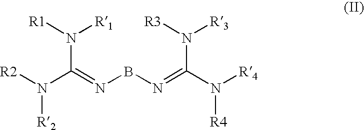
in which:
R1, R′₁, R2, R′₂, R3, R′₃, R4 and R′₄, which are identical or different, are each a group chosen from:
a hydrogen atom
a methyl, ethyl, propyl or isopropyl radical
R1 and R2 and/or R3 and R4 may also together form a divalent radical chosen from (CH₂)₂, (CH₂)₃ and CH═CH.
B is a radical chosen from: 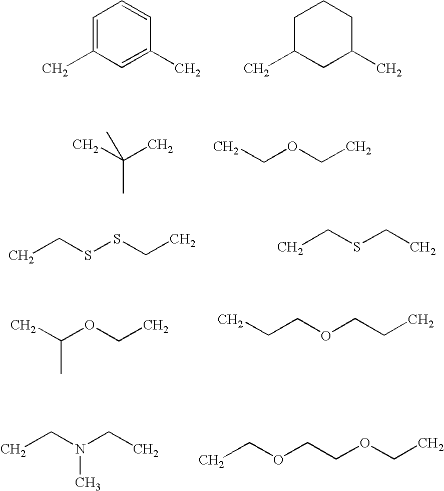
when R′₁═R′₂═R′₃═R′₄═CH₃ and when R1/R2 and R3/R4 respectively and simultaneously form the divalent radical (CH₂)₂:
B also is a radical (CH₂)₂ or (CH₂)₅ or (CH₂)₆ or a radical: 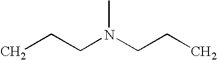
when R′₁═R′₂═R′₃═R′₄═CH₃ and when R1/R2 and R3/R4 respectively and simultaneously form the divalent radical (CH₂)₃:
B is (CH₂)₂ or (CH₂)₃ or (CH₂)₄ or (CH₂)₅ or (CH₂)₆ or a radical: 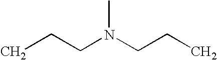
when R′₁═R′₂═R′₃═R′₄═CH₃ and when R1/R2 and R3/R4 respectively and simultaneously form the divalent radical CH═CH:
B is (CH₂)₃ or (CH₂)₄ or (CH₂)₅ or (CH₂)₆ or a radical: 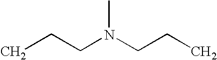
when R1, R′₁, R2, R′₂, R3, R′₃, R4 and R′₄ are independently chosen from ethyl and isopropyl radicals, B is a (CH₂)₃ or (CH₂)₄ or (CH₂)₅ or (CH₂)₆ radical or a radical 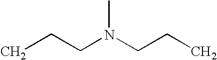
when R1, R′₁, R2, R′₂, R3, R′₃, R4 and R′₄ are each a methyl radical, B is a (CH₂)₄ or (CH₂)₅ radical.

15. The compound of formula (II) according to claim 14, wherein said compound is chosen from:
N,N,N′,N′-tetraisopropyl-N″-[3-(N′,N′,N″, N″-tetraisopropylguanidino)propyl]guanidine
N,N,N′,N′-tetraisopropyl-N″-[4-(N′,N′,N″,N″-tetraisopropylguanidino)butyl]guanidine
N,N,N′,N′-tetraisopropyl-N″-[5-(N′,N′,N″,N″-tetraisopropylguanidino)pentyl]guanidine
N,N,N′,N′-tetraisopropyl-N″-[6-(N′,N′,N″,N″-tetraisopropylguanidino)hexyl]guanidine
N,N,N′,N′-tetraisopropyl-N″-(3-{methyl-[3-(N′,N′,N″,N″-tetraisopropyl-guanidino)propyl]amino}propyl)guanidine
N,N,N′,N′-tetraethyl-N″-[3-(N′,N′,N″,N″-tetraethylguanidino)propyl]guanidine
N,N,N′,N′-tetraethyl-N″-[4-(N′,N′,N″,N″-tetraethylguanidino)butyl]guanidine
N,N,N′,N′-tetraethyl-N″-[5-(N′,N′,N″,N″-tetraethylguanidino)pentyl]guanidine
N,N,N′,N′-tetraethyl-N″-[6-(N′,N′,N″,N″-tetraethylguanidino)hexyl]guanidine
N,N,N′,N′-tetraethyl-N″-(3-{methyl-[3-(N′,N′,N″, N″-tetraisopropyl-guanidino)propyl]amino}propyl)guanidine
N,N′-bis(1,3-dimethylimidazolidin-2-ylidene)butane-1,4-diamine
N,N′-bis(1,3-dimethylimidazolidin-2-ylidene)pentane-1,5-diamine
N,N′-bis(1,3-dimethylimidazolidin-2-ylidene)hexane-1,6-diamine
N-(1,3-dimethylimidazolidin-2-ylidene)-N′-[3-(1,3-dimethylimidazolidin-2-ylideneamino)propyl]-N′-methylpropane-1,3,diamine
N,N′-bis(1,3-dimethyl-1,3-dihydroimidazol-2-ylidene)propane-1,3-diamine
N,N′-bis(1,3-dimethyl-1,3-dihydroimidazol-2-ylidene)butane-1,4-diamine
N,N′-bis(1,3-dimethyl-1,3-dihydroimidazol-2-ylidene)pentane-1,5-diamine
N,N′-bis(1,3-dimethyl-1,3-dihydroimidazol-2-ylidene)hexane-1,6-diamine
N-(1,3-dimethyl-1,3-dihydroimidazol-2-ylidene)-N′-[3-(1,3-dimethyl-1,3-dihydroimidazol-2-ylideneamino)propyl]-N′-methylpropane-1,3-diamine
N,N′-bis(1,3-dimethyltetrahydropyrimidin-2-ylidene)ethane-1,2-diamine
N,N′-bis(1,3-dimethyltetrahydropyrimidin-2-ylidene)propane-1,3-diamine
N,N′-bis(1,3-dimethyltetrahydropyrimidin-2-ylidene)butane-1,4-diamine
N,N′-bis(1,3-dimethyltetrahydropyrimidin-2-ylidene)pentane-1,5-diamine
N,N′-bis(1,3-dimethyltetrahydropyrimidin-2-ylidene)hexane-1,6-diamine
N-(1,3-dimethyltetrahydropyrimidin-2-ylidene)-N′-[3-(1,3-dimethyltetrahydropyrimidin-2-ylideneamino)propyl]-N′-methyl propane-1,3-diamine
N,N,N′,N′-tetramethyl-N″-[4-(N′,N′,N″,N″-tetramethylguanidino)butyl]guanidine
N,N,N′,N′-tetramethyl-N″-[5-(N′,N′,N″,N″-tetra-methylguanidino)pentyl]guanidine
N,N,N′,N′-tetramethyl-N″-[3-(N′,N′,N″,N″-tetra-methylguanidinomethyl)cyclohexylmethyl]guanidine
N,N,N′,N′-tetraethyl-N″-[3-(N′,N′,N″,N″-tetraethylguanidinomethyl)cyclohexyl-methyl]guanidine
N,N,N′,N′-tetrapropyl-N″-[3-(N′,N′,N″,N″-tetrapropylguanidinomethyl)cyclohexylmethyl]guanidine
N,N,N′,N′-tetraisopropyl-N″-[3-(N′,N′,N″,N″-tetra-isopropylguanidinomethyl)cyclohexylmethyl]guanidine
N,N,N′,N′-tetraethyl-N″-[3-(N′,N′,N″,N″-tetra-ethylguanidinomethyl)benzyl]guanidine
N,N,N′,N′-tetrapropyl-N″-[3-(N′,N′,N″,N″-tetra-propylguanidinomethyl)benzyl]guanidine
N,N,N′,N′-tetraisopropyl-N″-[3-(N′,N′,N″,N″-tetra-isopropylguanidinomethyl)benzyl]guanidine
N″-[2,2-dimethyl-3-(N′,N′,N″,N″-tetraethylguanidino)propyl]-N,N,N′,N′-tetraethylguanidine
N″-[2,2-dimethyl-3-(N′,N′,N″,N″-tetrapropylguanidino)propyl]-N,N,N′,N′-tetrapropylguanidine
N″-[2,2-dimethyl-3-(N′,N′,N″,N″-tetraisopropylguanidino)propyl]-N,N,N′,N′-tetraisopropylguanidine
N,N,N′,N′-tetramethyl-N″-{2-[2-(N′,N′,N″,N″-tetra-methylguanidino)ethoxy]ethyl}guanidine
N,N,N′,N′-tetraethyl-N″-{2-[2-(N′,N′,N″,N″-tetra-ethylguanidino)ethoxy]ethyl}guanidine
N,N,N′,N′-tetrapropyl-N″-{2-[2-(N′,N′,N″,N″-tetra-propylguanidino)ethoxy]ethyl}guanidine
N,N,N′,N′-tetraisopropyl-N″-{2-[2-(N′,N′,N″,N″-tetraisopropylguanidino)ethoxy]ethyl}guanidine
N,N,N′,N′-tetramethyl-N″-{2-[2-(N′,N′,N″,N″-tetramethylguanidino)ethyl-sulphanyl]ethyl}guanidine
N,N,N′,N′-tetraethyl-N″-{2-[2-(N′,N′,N″,N″-tetraethylguanidino)ethyl-sulphanyl]ethyl}guanidine
N,N,N′,N′-tetrapropyl-N″-{2-[2-(N′,N′,N″,N″-tetrapropylguanidino)ethyl-sulphanyl]ethyl}guanidine
N,N,N′,N′-tetraisopropyl-N″-{2-[2-(N′,N′,N″,N″-tetraisopropylguanidino)ethyl-sulphanyl]ethyl}guanidine
N,N,N′,N′-tetramethyl-N″-{2-[2-(N′,N′,N″,N″-tetramethylguanidino)ethyl-disulphanyl]ethyl}guanidine
N,N,N′,N′-tetraethyl-N″-{2-[2-(N′,N′,N″,N″-tetraethylguanidino)ethyl-disulphanyl]ethyl}guanidine
N,N,N′,N′-tetrapropyl-N″-{2-[2-(N′,N′,N″,N″-tetrapropylguanidino)ethyl-disulphanyl]ethyl}guanidine
N,N,N′,N′-tetraisopropyl-N″-{2-[2-(N′,N′,N″,N″-tetraisopropylguanidino)ethyl-disulphanyl]ethyl}guanidine
N,N,N′,N′-tetramethyl-N″-[2-methyl-5-(N′,N′,N″, N″-tetramethyl-guanidino)pentyl]guanidine
N,N,N′,N′-tetraethyl-N″-[2-methyl-5-(N′,N′,N″,N″-tetraethylguanidino)pentyl]-guanidine
N,N,N′,N′-tetrapropyl-N″-[2-methyl-5-(N′,N′,N″,N″-tetrapropylguanidino)pentyl]-guanidine
N,N,N′,N′-tetraisopropyl-N″-[2-methyl-5-(N′,N′,N″,N″-tetraisopropyl-guanidino)pentyl]guanidine
N,N,N′,N′-tetramethyl-N″-{3-[3-(N′,N′,N″,N″-tetramethylguanidino)propoxy]propyl}guanidine
N,N,N′,N′-tetraethyl-N″-{3-[3-(N′,N′,N″,N″-tetraethylguanidino)propoxy]propyl}guanidine
N,N,N′,N′-tetrapropyl-N″-{3-[3-(N′,N′,N″,N″-tetrapropylguanidino)propoxy]propyl}guanidine
N,N,N′,N′-tetraisopropyl-N″-{3-[3-(N′,N′,N″,N″-tetraisopropylguanidino)-propoxy]propyl}guanidine
N,N,N′,N′-tetramethyl-N″-(2-{methyl-[2-(N′,N′,N″,N″-tetramethylguanidino)-ethyl]amino}ethyl)guanidine
N,N,N′,N′-tetraethyl-N″-(2-{methyl-[2-(N′,N′,N″,N″-tetraethylguanidino)-ethyl]amino}ethyl)guanidine
N,N,N′,N′-tetrapropyl-N″-(2-{methyl-[2-(N′,N′,N″,N″-tetrapropylguanidino)-ethyl]amino}ethyl)guanidine
N,N,N′,N′-tetraisopropyl-N″-(2-{methyl-[2-(N′,N′, N″,N″-tetraisopropyl-guanidino)ethyl]amino}ethyl)guanidine
N,N,N′,N′-tetramethyl-N″-(2-{2-[2-(N′,N′,N″,N″-tetramethylguanidino)-ethoxy]ethoxy}ethyl)guanidine
N,N,N′,N′-tetraethyl-N″-(2-{2-[2-(N′,N′,N″,N″-tetraethylguanidino)-ethoxy]ethoxy}ethyl)guanidine
N,N,N′,N′-tetrapropyl-N″-(2-{2-[2-(N′,N′,N″,N″-tetrapropylguanidino)-ethoxy]ethoxy}ethyl)guanidine
N,N,N′,N′-tetraisopropyl-N″-(2-{2-[2-(N′,N′,N″,N″-tetraisopropylguanidino)-ethoxy]ethoxy}ethyl)guanidine
2-{3-[(1,3-dimethylimidazolidin-2-ylidene-amino)methyl]cyclohexylmethylimino}-1,3-dimethylimidazolidine
2-{3-[(1,3-dimethylimidazolidin-2-ylideneamino)methyl]benzylimino}-1,3-dimethylimidazolidine
N,N′-bis(1,3-dimethylimidazolidin-2-ylidene)-2,2-dimethylpropane-1,3-diamine
2-{2-[2-(1,3-dimethylimidazolidin-2-ylideneamino)ethylsulphanyl]ethylimino}-1,3-dimethylimidazolidine
2-{2-[2-(1,3-dimethylimidazolidin-2-ylideneamino)ethyldisulphanyl]ethylimino}-1,3-dimethylimidazolidine
N1,N5-bis(1,3-dimethylimidazolidin-2-ylidene)-2-methylpentane-1,5-diamine
N-(1,3-dimethylimidazolidin-2-ylidene)-N′-[2-(1,3-dimethylimidazolidin-2-ylideneamino)ethyl]-N′-methylethane-1,2-diamine
N1,N5-bis(1,3-dimethyl-1,3-dihydroimidazol-2-ylidene)-2-methylpentane-1,5-diamine
N-(1,3-dimethyl-1,3-dihydroimidazol-2-ylidene)-N′-[2-(1,3-dimethyl-1,3-dihydroimidazol-2-ylideneamino)ethyl]-N′-methylethane-1,2-diamine
2-{3-[(1,3-dimethyl-1,3-dihydroimidazol-2-ylidene-amino)methyl]cyclohexylmethylimino}-1,3-dimethyl-2,3-dihydro-1H-imidazole
N,N′-bis(1,3-dimethyl-1,3-dihydroimidazol-2-ylidene)-2,2-dimethylpropane-1,3-diamine
2-{3-[(1,3-dimethyl-1,3-dihydroimidazol-2-ylideneamino)methyl]benzyl imino}-1,3-dimethyl-2,3-dihydro-1H-imidazole
2-{2-[2-(1,3-dimethyl-1,3-dihydroimidazol-2-ylidene-amino)ethyldisulphanyl]ethylimino}-1,3-dimethyl-2,3-dihydro-1H-imidazole
2-{2-[2-(1,3-dimethyl-1,3-dihydroimidazol-2-ylidene-amino)ethylsulphanyl]ethylimino}-1,3-dimethyl-2,3-dihydro-1H-imidazole
N1,N5-bis(1,3-dimethyltetrahydropyrimidin-2-ylidene)-2-methylpentane-1,5-diamine
N-1,3-dimethyltetrahydropyrimidin-2-ylidene)-N′-[2-(1,3-dimethyltetra-hydropyrimidin-2-ylideneamino)ethyl]-N′-methylethane-1,2-diamine
2-{3-[(1,3-dimethyltetrahydropyrimidin-2-ylidene-amino)methyl]cyclohexylmethylimino}-1,3-dimethylhexahydropyrimidine
N,N′-bis(1,3-dimethyltetrahydropyrimidin-2-ylidene)-2,2-dimethylpropane-1,3-diamine
2-{3-[(1,3-dimethyltetrahydropyrimidin-2-ylideneamino)methyl]benzylimino}1,3-dimethylhexahydropyrimidine
2-{2-[2-(1,3-dimethyltetrahydropyrimidin-2-ylidene-amino)ethyldisulphanyl]ethylimino}-1,3-dimethylhexahydropyrimidine
2-{2-[2-(1,3-dimethyltetrahydropyrimidin-2-ylidene-amino)ethylsulphanyl]ethylimino}-1,3-dimethylhexahydropyrimidine

16. The compound of formula (II) according to claim 15, wherein the compound is chosen from:
N,N,N′,N′-tetraisopropyl-N″-[3-(N′,N′,N″, N″-tetra-isopropylguanidino)propyl]guanidine
N,N,N′,N′-tetraisopropyl-N″-[4-(N′,N′,N″, N″-tetra-isopropylguanidino)butyl]guanidine
N,N,N′,N′-tetraethyl-N″-[3-(N′,N′,N″,N″-tetraethylguanidino)propyl]guanidine
N,N,N′,N′-tetraethyl-N″-[4-(N′,N′,N″,N″-tetraethylguanidino)butyl]guanidine
N,N′-bis(1,3-dimethylimidazolidin-2-ylidene)butane-1,4-diamine
N-(1,3-dimethylimidazolidin-2-ylidene)-N′-[3-(1,3-dimethylimidazolidin-2-ylideneamino)propyl]-N′-methylpropane-1,3-diamine
N,N′-bis(1,3-dimethyl-1,3-dihydroimidazol-2-ylidene)propane-1,3-diamine
N,N′-bis(1,3-dimethyl-1,3-dihydroimidazol-2-ylidene)butane-1,4-diamine
N-(1,3-dimethyl-1,3-dihydroimidazol-2-ylidene)-N′-[3-(1,3-dimethyl-1,3-dihydroimidazol-2-ylideneamino)propyl]-N′-methylpropane-1,3-diamine
N,N′-bis(1,3-dimethyltetrahydropyrimidin-2-ylidene)ethane-1,2-diamine
N,N′-bis(1,3-dimethyltetrahydropyrimidin-2-ylidene)propane-1,3-diamine
N,N,N′,N′-tetramethyl-N″-[4-(N′,N′,N″,N″-tetramethylguanidino)butyl]guanidine
N,N,N′,N′-tetramethyl-N″-[3-(N′,N′, N″,N″-tetramethylguanidinomethyl)cyclo-hexylmethyl]guanidine
N,N,N′,N′-tetraethyl-N″-[3-(N′,N′,N″,N″-tetra-ethylguanidinomethyl)benzyl]guanidine
N″-[2,2-dimethyl-3-(N′,N′,N″,N″-tetraethylguanidino)propyl]-N,N,N′,N′-tetraethylguanidine
N,N,N′,N′-tetramethyl-N″-{2-[2-(N′,N′,N″,N″-tetra-methylguanidino)ethoxy]ethyl}guanidine
N,N,N′,N′-tetramethyl-N″-{2-[2-(N′,N′,N″,N″-tetramethylguanidino)ethyl-sulphanyl]ethyl}guanidine
N,N,N′,N′-tetramethyl-N″-{2-[2-(N′,N′,N″,N″-tetra-methylguanidino)ethyldisulphanyl]ethyl}guanidine
N,N,N′,N′-tetramethyl-N″-[2-methyl-5-(N′,N′,N″,N″-tetramethylguanidino)pentyl]guanidine
N,N,N′,N′-tetramethyl-N″-{3-[3-(N′,N′,N″,N″-tetra-methylguanidino)propoxy]propyl}guanidine
N,N,N′,N′-tetramethyl-N″-(2-{methyl-[2-(N′,N′,N″,N″-tetramethylguanidino)-ethyl]amino}ethyl)guanidine
N,N,N′,N′-tetramethyl-N″-(2-{2-[2-(N′,N′,N″,N″-tetramethylguanidino)ethoxy]ethoxy}ethyl)guanidine
2-{3-[(1,3-dimethylimidazolidin-2-ylidene-amino)methyl]cyclohexylmethylimino}-1,3-dimethylimidazolidine
2-{2-[2-(1,3-dimethylimidazolidin-2-ylideneamino)ethylsulphanyl]ethylimino}-1,3-dimethylimidazolidine
N-(1,3-dimethyl-1,3-dihydroimidazol-2-ylidene)-N′-[2-(1,3-dimethyl-1,3-dihydroimidazol-2-ylideneamino)ethyl]-N′-methylethane-1,2-diamine
2-{2-[2-(1,3-dimethyl-1,3-dihydroimidazol-2-ylidene-amino)ethyldisulphanyl]ethylimino}-1,3-dimethyl-2,3-dihydro-1H-imidazole
N1, N5-bis(1,3-dimethyltetrahydropyrimidin-2-ylidene)-2-methylpentane-1,5-diamine
2-{3-[(1,3-dimethyltetrahydropyrimidin-2-ylideneamino)methyl]benzylimino}-1,3-dimethylhexahydropyrimidine

17. The cosmetic composition according to claim 1, wherein the cosmetic composition also comprises at least one adjuvant chosen from silicones in soluble, dispersed or microdispersed form, nonionic, anionic, cationic and amphoteric surfactants, ceramides, glycoceramides and pseudoceramides, vitamins and provitamins, plant, animal, mineral and synthetic oils, waxes other than ceramides, glycoceramides and pseudoceramides, water-soluble and liposoluble, silicone-based or non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents.

18. A multi-compartment kit comprising at least two compartments, one of the compartments (i) comprising, in a cosmetically acceptable medium, at least one polyguanidine not belonging to the hydroxide family and capable of reacting with the cystines of keratin fibers, via a beta-elimination reaction producing dehydroalanine and leading to the formation of lanthionine, to relax the keratin fibers in less than 60 minutes.

19. The multi-compartment kit according to claim 18, wherein the multi-compartment kit further comprises an additional composition (ii) for caring for, conditioning, making up, removing makeup from, protecting, cleansing or washing keratin fibers.

20. A process for relaxing keratin fibers, said process comprising
applying to said keratin materials a cosmetic composition comprising, in a cosmetically acceptable medium, at least one polyguanidine not belonging to the hydroxide family, the cosmetically acceptable medium and the polyguanidine being chosen such that the polyguanidine not belonging to the hydroxide family reacts on the cystines of the keratin fibers, via a beta-elimination reaction producing dehydroalanine and leads to the formation of lanthionine, relaxing the keratin fibers in less than 60 minutes.

21. The process for relaxing keratin fibers according to claim 20, further comprising, after application of the composition, subjecting the keratin fibers to a heat treatment by heating to a temperature ranging from 30 to 60° C.

22. The process for relaxing keratin fibers according to claim 20, comprising heating and smoothing out said keratin fibers with a heating iron at a temperature ranging from 60 to 220° C.

23. The process for relaxing keratin fibers according to claim 22, wherein said heating iron is at a temperature ranging from 120 to 200° C.

24. The process for relaxing keratin fibers according to claim 20, wherein the relaxing time is less than 40 minutes.

25. The process for relaxing keratin materials according to claim 24, wherein the relaxing time is less than 30 minutes.

26. A process for relaxing keratin fibers, said process comprising
applying to said keratin materials a cosmetic composition comprising, in a cosmetically acceptable medium, at least one compound of formula (II): 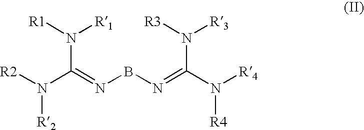
in which:
R1, R′₁, R2, R′₂, R3, R′₃, R4 and R′₄, which are identical or different, are each a group chosen from:
a hydrogen atom
a methyl, ethyl, propyl or isopropyl radical
R1 and R2 and/or R3 and R4 may also together form a divalent radical chosen from (CH₂)₂, (CH₂)₃ and CH═CH.
B is a radical chosen from: 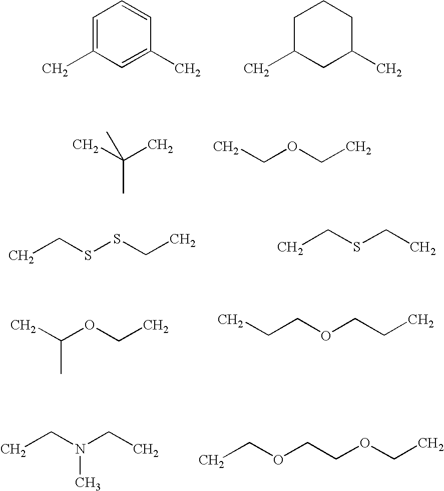
when R′₁═R′₂═R′₃═R′₄═CH₃ and when R1/R2 and R3/R4 respectively and simultaneously form the divalent radical (CH₂)₂:
B also is a radical (CH₂)₂ or (CH₂)₅ or (CH₂)₆ or a radical: 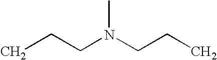
when R′₁═R′₂═R′₃═R′₄═CH₃ and when R1/R2 and R3/R4 respectively and simultaneously form the divalent radical (CH₂)₃:
B is (CH₂)₂ or (CH₂)₃ or (CH₂)₄ or (CH₂)₅ or (CH₂)₆ or a radical: 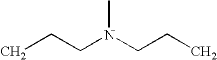
when R′₁═R′₂═R′₃═R′₄═CH₃ and when R1/R2 and R3/R4 respectively and simultaneously form the divalent radical CH═CH:
B is (CH₂)₃ or (CH₂)₄ or (CH₂)₅ or (CH₂)₆ or a radical: 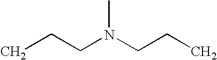
when R1, R′₁, R2, R′₂, R3, R′₃, R4 and R′₄ are independently chosen from ethyl and isopropyl radicals, B is a (CH₂)₃ or (CH₂)₄ or (CH₂)₅ or (CH₂)₆ radical or a radical 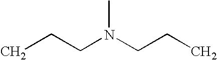
when R1, R′₁, R2, R′₂, R3, R′₃, R4 and R′₄ are each a methyl radical, B is a (CH₂)₄ or (CH₂)₅ radical,
wherein said composition comprising at least one compound of formula (II) reacts on the cystines of the keratin fibers via a beta-elimination reaction producing dehydroalanine and leads to the formation of lanthionine, relaxing the keratin fibers in less than 60 minutes.
